(19) United States
(12) Patent Application Publication
(10) Pub. No.: US 2009/0056480 A1
(43) Pub. Date: Mar. 5, 2009

(54) APPARATUS AND METHOD FOR DETERMINING MINERAL CONTENT

(76) Inventor: Toshie Ishitani, Dewinton (CA)

Correspondence Address: Maxey Law Offices, PLLC, 13630 58TH ST. NORTH, SUITE 101, CLEARWATER, FL, 33760, US

(21) Appl. No.: 11/849,382

(22) Filed: Sep. 4, 2007

(51) Int. Cl. G01N 33/24

(52) U.S. Cl. 73/866

(57) ABSTRACT

Determination of the mineral content of a sample by suspending a needle at its center of mass horizontally above the sample is provided. In doing this, the needle will rotate or swing back and forth along an arc of defined starting and ending positions in a horizontal plane. Samples of different mineral content will cause the needle to swing back and forth along a similar arc but with different defined starting and ending positions. By recording the starting and ending positions of the arc for known samples a chart is compiled that can be used to make a determination of the mineral content of unknown samples. An apparatus and method of using the same is also provided.

### BACKGROUND OF THE INVENTION

### Field of the Invention

The present invention relates generally to a scientific instrument and method of using the same, and more particularly, relating to a scientific instrument and method of using the same for determining mineral content of material samples.

### SUMMARY OF THE INVENTION

I have discovered you can make a determination of the largest mineral content of a sample by suspending a needle at its center of mass horizontally above the sample. In doing this, I have discovered the needle will rotate or swing back and forth along an arc of defined starting and ending positions in a horizontal plane. Samples of different mineral content will cause the needle to swing back and forth along a similar arc but with different defined starting and ending positions. By recording the starting and ending positions of the arc for known samples, I have compiled a chart that can be used to make a determination of the mineral content of unknown samples. I have also devised an apparatus and a method of using the same for determining the mineral content of samples.

In general, in one aspect, method for determining the mineral content of a sample is provided. The method includes the steps of:
providing a needle suspended from above at its center of mass by a support;
providing a 360-degree protractor;
providing a look-up chart;
providing a sample to be tested;
positioning the 360-degree protractor below said needle such that the 0-degree mark is aligned with said needle;
positioning the sample below said needle;
observing the rotation arc of the needle along the 360-degree protractor;
recording the angular degree of the starting position and ending position of the rotation arc of the needle; and
determining mineral content of the sample by looking-up the rotation arc starting and ending positions on the look-up chart.

In general, in another aspect, an apparatus for determining the mineral content of a sample. The apparatus includes in combination:
a support;
a needle suspended at its center of mass by the support such that its free to rotate 360-degrees in the horizontal plane; and
a 360-degree protractor rotatably positioned below the support.

There has thus been outlined, rather broadly, the more important features of the invention in order that the detailed description thereof that follows may be better understood and in order that the present contribution to the art may be better appreciated.

Numerous objects, features and advantages of the present invention will be readily apparent to those of ordinary skill in the art upon a reading of the following detailed description of presently preferred, but nonetheless illustrative, embodiments of the present invention when taken in conjunction with the accompanying drawings. The invention is capable of other embodiments and of being practiced and carried out in various ways. Also, it is to be understood that the phraseology and terminology employed herein are for the purpose of descriptions and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

For a better understanding of the invention, its operating advantages and the specific objects attained by its uses, reference should be had to the accompanying drawings and descriptive matter in which there is illustrated preferred embodiments of the invention.

### BRIEF DESCRIPTION OF THE DRAWINGS

The invention will be better understood and objects other than those set forth above will become apparent when consideration is given to the following detailed description thereof. Such description makes reference to the annexed drawings wherein:

FIG. 1 is a diagrammatic perspective view of the apparatus constructed in accordance with the principles of the present invention;

FIG. 2 is a diagrammatic top view of the apparatus, illustrating the initial at rest position of the needle and an example of the starting and ending positions of an arc the needle rotates back and forth between for sample; and

FIG. 3 is a diagrammatic view of an exemplary chart of known minerals and the respective starting and ending positions of the arc through which the needle rotates back and forth between.

### DETAILED DESCRIPTION OF THE INVENTION

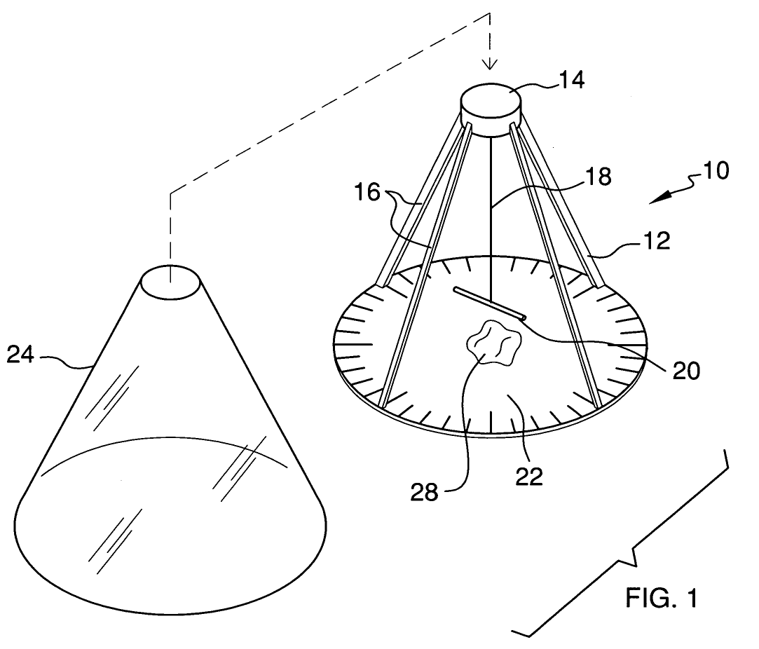

Referring now to FIGS. 1-3 of the drawings, reference numeral 10 generally designates the apparatus for determining mineral content of a sample of the present invention.

In FIG. 1, the apparatus includes a support 12 having a top portion 14 which is elevated by a plurality of support legs 16 of equal lengths. A string 18 of a determinate length depends centrally from the bottom of the top portion 14. A needle 20 is suspended horizontally at its center of mass by the string 18 such that the needle is free to rotate in the horizontal plane. The needle 20 is of a metallic material and is not magnetized. A 360-degree protractor 22 is rotatably positioned bellow the needle. The support 12 can include a removable at least partially transparent covering 24 or it can be integrally formed therewith.

The apparatus 10 is initially setup allowing the suspended needle 20 to come to rest and then rotating the 360-degree protractor so as to zero the protractor with the needle. In other words, the protractor 22 is rotated such that the 0-degree hash mark is in alignment with the end of the needle 20 that is point towards magnetic north.

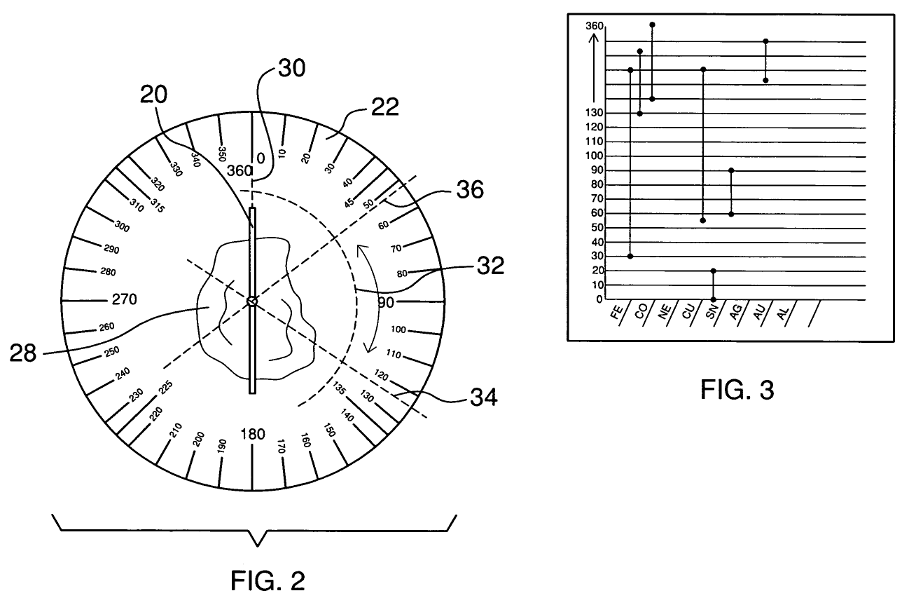

With reference to FIG. 2, once setup, a material sample 28 is positioned directly below the needle 20. The needle 20 will then begin to rotate from the at rest position 30 and will continue to rotate along an arc 32 until reaching an end point 34. Once end point 34 has been reached, the needle 20 will reverse its rotation and rotate in a direction back towards the at rest position 30 until it reaches a second end point 36 along the arc 32, which can be any point along the arc. Once end point 36 is reached, the needle 20 will again reverse its rotation and rotate along the arc 32 until reaching the end point 34. The needle 20 will continue to swing or rotate back and forth between the first and second end points 34 and 36 respectively. The first and second end points 34 and 36 are recorded using the 360-degree protractor.

I have discovered different minerals will cause the needle 20 to rotate along distinct and different arcs. Taking known minerals, a chart of the arc length and end points of each mineral can be compiled allowing the testing of unknown samples. FIG. 3 illustrates an example of such a chart. The following table is an example of several arc lengths and end points for known minerals I have tested:

| | First Point - | Second Point - | Arc Length - |
|---|---|---|---|
| Mineral | degrees | degrees | degrees |
| Iron (Fe) | 20-degrees | 160-degrees | 140-degrees |
| Cobalt (Co) | 130-degrees | 170-degrees | 140-degrees |
| Nickel (Ne) | 140-degrees | 220-degrees | 80-degrees |
| Copper (Cu) | 55-degrees | 160-degrees | 105-degrees |
| Tin (Sn) | 0-degrees | 20-degrees | 20-degrees |
| Silver (Ag) | 60-degrees | 95-degrees | 35-degrees |
| Gold (Au) | 160-degrees | 270-degrees | 110-degrees |
| Aluminum (Al) | 160-degrees | 180-degrees | 20-degrees |

The above table illustrates a finite number of minerals that I have sampled with the apparatus 10. One could compile a similar chart by first testing known minerals, which results can then be used to test unknown samples.

A number of embodiments of the present invention have been described. Nevertheless, it will be understood that various modifications may be made without departing from the spirit and scope of the invention. Accordingly, other embodiments are within the scope of the following claims.

1. A method for determining the mineral content of a sample, the method comprising the steps of:
providing a needle suspended from above at its center of mass by a support;
providing a 360-degree protractor;
positioning the 360-degree protractor below said needle such that the 0-degree mark is aligned with said needle and said needle is pointing to the 0-degree mark;
positioning a sample to be tested below the needle;
observing the rotation arc of the needle along the 360-degree protractor;
recording the angular degree of the starting position and ending position of the rotation arc of the needle; and
determining mineral content of the sample by looking-up the rotation arc starting and ending positions on a look-up chart.

2. A method for determining the mineral content of a sample, the method comprising the steps of:
providing a needle suspended from above at its center of mass by a support having clear sidewalls which encloses the needle;
providing a 360-degree protractor;
providing a sample to be tested;
positioning the 360-degree protractor below the needle such that the 0-degree mark is aligned with said needle and said needle is pointing to the 0-degree mark;
positioning a sample to be tested below the needle;
observing the rotation arc of the needle along the 360-degree protractor;
recording the angular degree of the starting position and ending position of the rotation arc of the needle; and
determining mineral content of the sample by looking-up the rotation arc starting and ending positions on a look-up chart.

3. An apparatus for determining the mineral content of a sample, the apparatus comprising in combination:
a support;
a needle suspended at its center of mass by said support such that its free to rotate 360-degrees in the horizontal plane; and
a 360-degree protractor rotatably positioned below said support and said needle.

4. The apparatus of claim 3, wherein said support includes clear sidewalls which enclose said needle.

5. The apparatus of claim 4, wherein said needle is suspended by said support by a thread.

6. The method of claim 1, wherein said needle is of a metallic material and is non-magnetic.

7. The method of claim 2, wherein said needle is of a metallic material and is non-magnetic.
